(19) United States
(12) Patent Application Publication
(10) Pub. No.: US 2023/0149173 A1
(43) Pub. Date: May 18, 2023

(54) BONE IMPLANT

(71) Applicant: Aesculap AG, Tuttlingen (DE)

(72) Inventors: Elvira Stolinski, Messkirch (DE); Marc Baxmann, Harsum (DE); Denis Hoeffgen, Engen (DE); Georg Hettich, Freiburg (DE); Thomas Grupp, Denkingen (DE)

(21) Appl. No.: 17/918,028

(22) PCT Filed: Apr. 23, 2021
(86) PCT No.: PCT/EP2021/060632

§ 371 Date: Oct. 10, 2022

(30) Foreign Application Priority Data
Apr. 24, 2020 (DE) 10 2020 205 229.8

(51) Int. Cl. A61F 2/30

(52) U.S. Cl. CPC A61F 2/30771; A61F 2002/30064; A61F 2002/30894; A61F 2002/3079; A61F 2002/30593; A61F 2002/30217; A61F 2002/30032; A61F 2002/30784; A61F 2002/30838; A61F 2002/30985; A61F 2002/30112; A61F 2002/3092

(57) ABSTRACT

A bone implant includes a main body in the form of a hollow body open on both sides in the axial direction. The main body includes a load-bearing material. An encasing body at least partially encases the main body on the outside and includes an in vivo degradable/in vivo resorbable material. Alternatively, the encasing body includes a multiplicity of shaped bodies protruding from the main body in the radial direction that include an in vivo degradable/in vivo resorbable material. A method for producing the bone implant includes an additive manufacturing process. The main body can be at least partially encased by the encasing body in the additive manufacturing process.

### CROSS-REFERENCE TO RELATED APPLICATIONS

This application is the United States national stage entry of International Application No. PCT/EP2021/060632, filed Apr. 23, 2021, and claims priority to German Application No. 10 2020 205 229.8, filed Apr. 24, 2020. The contents of International Application No. PCT/EP2021/060632 and German Application No. 10 2020 205 229.8 are incorporated by reference herein in their entireties.

### FIELD

The invention relates to a bone implant, especially for treating or managing bone defects and/or for anchoring or fixing a joint implant, preferably a knee joint implant.

### BACKGROUND

In general, complex bone defects in revision surgery on a joint are a major challenge. This is especially the case when treating or managing complex tibial or femoral bone defects. Treating or managing such bone defects is made even more difficult by the fact that the shape and/or geometry of the tibia (shinbone) and femur (thighbone) is subject to natural variation depending on the height, sex and ethnic origin of a patient.

The treatment of complex joint bone defects generally involves pursuing two primary goals, namely stable anchoring of a bone implant, used for treating or managing the bone defect, in a bony bed with high primary stability, and reconstruction of the original biomechanical axes.

Conventional implants generally have the disadvantage that they only reproduce the morphological conditions, in particular the shape and/or the geometry and/or the proportions, of bone defects, in particular tibial or femoral bone defects, to a limited extent. A consequence of this is that the shape and/or the geometry and/or the size of the bone defect has to be adapted to the particular implant in order to achieve optimal cortical support. The associated (additional) bone loss in turn creates unfavorable starting conditions with respect to any necessary revision surgery.

### SUMMARY

It is an object of the invention to provide a bone implant which completely or partially avoids the disadvantages described at the beginning in connection with conventional bone implants. In particular, the bone implant shall make it possible to treat or manage bone defects in a manner that is gentle on bone and shall reduce the risk of revision surgery.

According to a first aspect, the invention provides a bone implant, preferably for treating or managing bone defects, in particular tibial and/or femoral bone defects, and/or for fixing or anchoring a joint implant, in particular a knee joint implant.

The bone implant comprises the following:
a main body in the form of a hollow body open on both sides in the axial direction, comprising or consisting of a load-bearing material,
and
an encasing body which at least partially, in particular only partially or completely, encases the main body or hollow body on the outside, i.e., on an outer face of the main body or hollow body, and comprises an in vivo degradable/in vivo resorbable material or consists of an in vivo degradable/in vivo resorbable material, or a multiplicity of shaped bodies which protrude from the main body or hollow body in the radial direction and comprise an in vivo degradable/in vivo resorbable material or consist of an in vivo degradable/in vivo resorbable material.

The expression “bone implant” is to be understood in the context of the present invention to mean an implant for treating or managing a bone defect, in particular a tibial or femoral bone defect, and/or for fixing or anchoring a joint implant, in particular a knee joint implant.

The expression “shaped body” is to be understood in the context of the present invention to mean bodies or components of the bone implant that are designed to fix, anchor or support the main body or hollow body on/in a bone defect, in particular on/in a tibial or femoral bone defect.

The expression “multiplicity of shaped bodies” is to be understood in the context of the present invention to mean at least two, in particular more than two, shaped bodies, for example two, three, four, five, six, seven, eight, nine, ten or more shaped bodies.

The expression “bone defect” in the context of the present invention means a bone area, in particular joint bone area, preferably knee or hip joint bone area, that is affected by loss of or damage to bone tissue, in particular joint bone tissue, preferably knee or hip joint bone tissue. Bone loss or bone damage can be the result of a bone fracture, bone trauma, necrosis, thinning (e.g., as a result of osteoporosis), a bone disease such as a tumor disease, a lack of mechanical stress (so-called stress shielding) or the result of surgical intervention/reintervention, in particular a revision, preferably after a total knee or hip joint replacement.

The expression “in vivo degradable/in vivo resorbable” in the context of the present invention means in vivo degradable or in vivo resorbable.

The expression “non-in vivo degradable/non-in vivo resorbable” in the context of the present invention means non-in vivo degradable or non-in vivo resorbable.

The expression “vivo degradation rate/in vivo resorption rate” in the context of the present invention means in vivo degradation rate or in vivo resorption rate.

The expression “load-bearing material” is to be understood in the context of the present invention to mean a material capable of withstanding forces which usually occur in a bone, such as in particular a shinbone and/or thighbone, without deformation or destruction or without significant deformation or destruction.

The expression “in vivo degradable material” is to be understood in the context of the present invention to mean a material which can be metabolized in a human or animal body, in particular under the action of enzymes. The degradation of the material can proceed all the way to mineralization, i.e., the release of chemical elements and the incorporation thereof into inorganic compounds, such as, for example, carbon dioxide and/or oxygen and/or ammonia, or remain at the stage of intermediates or transformation products that are degradation-stable.

The expression “in vivo resorbable material” is to be understood in the context of the present invention to mean a material which can be taken up in a human or animal body by living cells or living tissue, such as, for example, kidneys, without degradation or appreciable degradation of the material taking place.

The expression “animal body” is to be understood in the context of the present invention to mean the body of a nonhuman mammal, such as, for example, horse, cow, goat, sheep, pig or a rodent, such as, for example, leporid, rat or mouse.

The expression “in the axial direction” is to be understood in the context of the present invention to mean along an axis, in particular an axis of symmetry, of a component or a body, such as, for example, the main body or hollow body and/or the encasing body of the bone implant.

The expression “longitudinal axis” is to be understood in the context of the present invention to mean an axis of a component or a body, such as, for example, the main body or hollow body and/or the encasing body of the bone implant, that corresponds to the direction of the greatest extent of the component or the body, such as, for example, the main body or hollow body and/or the encasing body.

The expression “in the radial direction” is to be understood in the context of the present invention to mean pointing away from a point of symmetry, in particular center point, and/or from an axis of symmetry, in particular central axis, of a component or a body, such as, for example, the main body or hollow body and/or the encasing body of the bone implant.

The expression “proximal” is to be understood in the context of the present invention to mean pointing toward the center of the body or located closer to the center of the body.

The expression “distal” is to be understood in the context of the present invention to mean pointing away from the center of the body or located away from the center of the body.

The expression “conical hollow body” is to be understood in the context of the present invention to mean a hollow body which has a side or a lateral face, wherein the side or the lateral face is at least sectionally, in particular only sectionally or completely, inclined with respect to an axis of symmetry, in particular central axis, of the hollow body.

The invention is distinguished in particular by the following advantages:
Owing to the load-bearing material of the main body or hollow body, a medically sufficient primary and secondary stability is achievable with particular advantage. Primary stability is generally understood to mean the fixation of an implant in the first few weeks after an operation, which fixation is based on friction or fastening elements, such as, for example, bone screws. Secondary stability is understood to mean the fixation of an implant that is based on bony adhesions. Secondary stability generally sets in about a month after a surgical procedure and can last up to several years.
Furthermore, the in vivo degradable/in vivo resorbable material of the encasing body or the shaped bodies advantageously allows biological reconstruction of a bone defect and thus size reduction of the bone defect. As a result, it is possible in particular to create improved starting conditions for any revision surgery.
A further advantage is that selecting the in vivo degradable/in vivo resorbable material of the encasing body or the shaped bodies makes it possible to specifically control the mechanical stability of the bone implant and to specifically influence bone stimulation and bone growth. In particular, this can balance the mechanical stability of the bone implant, bone stimulation and bone growth in relation to one another.
The bone implant according to the present invention is especially suitable for treating and/or managing tibial and/or femoral bone defects.

In one embodiment of the invention, the main body or hollow body has a corner-free, in particular circular, oval or elliptical, cross section.

In a further embodiment of the invention, the main body or hollow body is conical. A conical main body or hollow body has, in particular, the advantage of allowing more stable anchoring of an extension shaft of a joint implant. Preferably, the main body or hollow body has a tapering cross-sectional area, in particular a continuously or discontinuously tapering cross-sectional area, and/or a tapering inner diameter, in particular a continuously or discontinuously tapering inner diameter, in the axial direction or longitudinal direction.

Alternatively, the main body or hollow body can have a constant cross-sectional area and/or a constant inner diameter in the axial direction or longitudinal direction, in particular throughout said direction.

In principle, the main body or hollow body can have a constant, i.e., uniform, or a varying, i.e., nonuniform, wall thickness, in particular in the axial direction or longitudinal direction. As a result, it is possible with particular advantage to accommodate asymmetrical bone defects.

In a further embodiment of the invention, the main body or hollow body has a wall thickness of 1 mm to 30 mm, in particular 5 mm to 20 mm, preferably 8 mm to 15 mm.

Furthermore, the main body or hollow body can have a constant, i.e., uniform, or a varying, i.e., nonuniform, shape, in particular in the axial direction or longitudinal direction. As a result, it is (also) possible with particular advantage to accommodate asymmetrical bone defects.

Furthermore, the main body or hollow body can have a wall having a number of openings or cutouts, i.e., only one opening/one cutout or a multiplicity of openings/cutouts, in the axial direction, in particular in the longitudinal direction. The opening/cutout or the openings/cutouts can, in particular, be U-shaped. Preferably, the wall of the main body or hollow body has two openings/cutouts, in particular two opposing openings/cutouts. As a result, it is possible with particular advantage to position a knee joint implant in the main body or hollow body. Tibial components of knee joint implants namely have mediolaterally attached “wings” for maintaining stable anchoring and rotational stability.

In a further embodiment of the invention, the load-bearing material of the main body or hollow body is an in vivo nondegradable/in vivo nonresorbable material. As a result, a particularly effective primary and secondary stability of the bone implant is realizable with particular advantage.

The in vivo nondegradable/in vivo nonresorbable material is preferably selected from the group consisting of metals, alloys, ceramics, plastics and combinations, in particular mixtures, thereof.

The metal can, in particular, be selected from the group consisting of tantalum, titanium, iron, zirconium, niobium, gold and platinum. Particular preference is given to titanium.

The alloy can, in particular, be selected from the group consisting of magnesium alloy, tantalum alloy, titanium alloy, chromium-cobalt alloy, chromium-cobalt-molybdenum alloy, iron alloy, steel such as stainless steel, zirconium alloy, niobium alloy, gold alloy, platinum alloy and combinations, in particular mixtures, thereof. Particular preference is given to a titanium alloy, in particular Ti-6Al-4V.

The plastic can, in particular, be selected from the group consisting of polyetherketones, polyolefins, polyesters, polyamides, copolymers and combinations, in particular mixtures (blends), thereof.

The polyetherketones can, in particular, be selected from the group consisting of polyetherketone (PEK), polyetheretherketone (PEEK), polyetherketoneketone (PEKK), polyetheretheretherketone (PEEEK), polyetheretherketoneketone (PEEKK), polyetherketoneetherketoneketone (PEKEKK), copolymers thereof and combinations, in particular mixtures (blends), thereof. Particular preference is given to polyetheretherketone (PEEK).

The polyolefins can, in particular, be selected from the group consisting of polyethylene (PE), low density polyethylene, high density polyethylene, high molecular weight polyethylene (HMWPE), ultrahigh molecular weight polyethylene (UHMWPE), polypropylene (PP), polytetrafluoroethylene (PTFE), polyvinylidene fluoride, polyvinylidene chloride, polytetrafluoropropylene, polyhexafluoropropylene, copolymers thereof and combinations, in particular mixtures (blends), thereof.

The polyesters can, in particular, be selected from the group consisting of polyethylene terephthalate, polypropylene terephthalate, polybutylene terephthalate, copolymers and combinations, in particular mixtures (blends), thereof.

In a further embodiment of the invention, the encasing body is in the form of a layer or coating on the main body or hollow body.

In a further embodiment of the invention, the encasing body has a thickness of 0.1 mm to 30 mm, in particular 1 mm to 10 mm, preferably 2 mm to 5 mm.

Furthermore, the encasing body can have a constant, i.e., uniform, thickness and/or a constant, i.e., uniform, shape, in particular in the axial direction or longitudinal direction.

In a further embodiment of the invention, the encasing body has a varying, i.e., nonuniform, shape and/or thickness, in particular in the axial direction or longitudinal direction. As a result, it is (likewise) possible with particular advantage to accommodate asymmetrical bone defects.

In a further embodiment of the invention, the thickness of the encasing body is greater at one end of the encasing body than another end of the encasing body, in particular in the axial direction or longitudinal direction, and/or the thickness of the encasing body increases, in particular continuously or discontinuously, toward one end of the encasing body, in particular in the axial direction or longitudinal direction, and/or the thickness of the encasing body increases, in particular continuously or discontinuously, from one end of the encasing body to another end of the encasing body, in particular in the axial direction or longitudinal direction.

If the bone implant is a tibial implant, i.e., an implant for treating or managing a tibial bone defect, the thickness of the encasing body is preferably greater at a distal end of the encasing body than at a proximal end of the encasing body, in particular in the axial direction or longitudinal direction, and/or the thickness of the encasing body preferably increases, in particular continuously or discontinuously, toward a distal end of the encasing body, in particular in the axial direction or longitudinal direction, and/or the thickness of the encasing body preferably increases, in particular continuously or discontinuously, from a proximal end of the encasing body to a distal end of the encasing body, in particular in the axial direction or longitudinal direction.

If the bone implant is a femoral implant, i.e., an implant for treating or managing a femoral bone defect, the thickness of the encasing body is preferably greater at a proximal end of the encasing body than at a distal end of the encasing body, in particular in the axial direction or longitudinal direction, and/or the thickness of the encasing body preferably increases, in particular continuously or discontinuously, toward a proximal end of the encasing body, in particular in the axial direction or longitudinal direction, and/or the thickness of the encasing body preferably increases, in particular continuously or discontinuously, from a distal end of the encasing body to a proximal end of the encasing body, in particular in the axial direction or longitudinal direction.

In a further embodiment of the invention, the main body or hollow body is partially not surrounded by the encasing body on the outside.

In a further embodiment of the invention, the main body or hollow body has a porous, in particular open-pored, and/or microstructured surface. This can induce osteointegration, i.e., take or ingrowth of natural bone tissue. This in turn promotes, with particular advantage, stable fixation or anchoring of the main body and thus of the bone implant on/in a bone defect. As a result, improved secondary stabilities are achievable in particular. Furthermore, the take or ingrowth of bone tissue advantageously brings about a reduction in bone defect size.

Preferably, the porous, in particular open-pored, surface of the main body or hollow body has pores having a diameter, in particular average diameter, determined by means of scanning electron microscopy and/or mercury porosimetry, of 10 μm to 400 μm, in particular 50 μm to 200 μm, preferably 100 μm to 135 μm. The pore diameters disclosed in this paragraph are particularly advantageous with respect to osteointegration of the main body or hollow body.

Furthermore, pores, in particular surface pores, of the main body or hollow body can be filled with an in vivo degradable/in vivo resorbable material. The pores can, in particular, be only partially or completely filled with the in vivo degradable/in vivo resorbable material. Preferably, the in vivo degradable/in vivo resorbable material which fills the pores, in particular surface pores, of the main body or hollow body and the in vivo degradable/in vivo resorbable material of the enclosing body or the multiplicity of shaped bodies differ from one another. As a result, multistage, for example two-stage, degradation or resorption kinetics of the bone implant can be generated. This is advantageously a further way of balancing the mechanical stability of the bone implant, bone stimulation and bone growth in relation to one another. With regard to suitable in vivo degradable/in vivo resorbable materials, reference is made to the in vivo degradable/in vivo resorbable materials disclosed in the description that follows in connection with the encasing body and the multiplicity of shaped bodies.

The microstructured surface of the main body or hollow body can, for example, be formed by a metal structure, in particular metal lattice structure. Preferably, the metal structure, in particular metal lattice structure, is a titanium structure, in particular titanium lattice structure. A relevant titanium structure is, for example, commercially available under the registered trademark STRUCTAN®. With regard to further suitable materials for the main body or hollow body, reference is made to the above materials described in connection with the main body or hollow body.

The expression “microstructured surface” is to be understood in the context of the present invention to mean a surface having structural elements, in particular in the form of depressions and/or elevations, said structural elements having dimensions, for example depths and/or heights, on a micrometer scale.

For example, the microstructured surface of the main body or hollow body can have depressions with a depth of 20 μm to 1000 μm, in particular 100 μm to 600 μm, preferably 300 μm to 400 μm, and/or elevations with a height of 20 μm to 1000 μm, in particular 100 μm to 600 μm, preferably 300 μm to 400 μm.

Furthermore, the surface of the main body or hollow body can be formed by a coating of the main body or hollow body. In other words, the surface of the main body or hollow body can be a surface of a coating which coats or covers the main body or hollow body. The coating can at least partially, in particular only partially or completely, coat or cover the main body or hollow body. Furthermore, the coating can have a thickness of 0.01 μm to 1000 μm, in particular 1 μm to 100 μm, preferably 10 μm to 50 μm. Furthermore, the coating can have a porosity of 20% to 90%, in particular 50% to 85%, preferably 60% to 80%. Preferably the coating comprises titanium, i.e., elemental or metallic titanium, or a calcium phosphate, preferably hydroxyapatite. For example, the coating can be a pure titanium coating, i.e., a coating consisting of titanium. A relevant coating can, for example, be produced by means of a process carried out by the applicant under the registered trademark PLASMAPORE®. Here, pure titanium powder is applied to a substrate to be coated with a thickness of 150 μm and a porosity of 35% to 60% by means of a plasma coating process under vacuum or negative pressure. With regard to further suitable materials for the coating, reference is made to the above materials described in connection with the main body or hollow body.

In a further embodiment of the invention, the encasing body has a porous, in particular open-pored, and/or microstructured surface. This can (likewise) induce osteointegration and improve the secondary stability of the bone implant and, depending on the material of the encasing body, achieve a reduction in bone defect size.

Preferably, the porous, in particular open-pored, surface of the encasing body has pores having a diameter, in particular average diameter, determined by means of scanning electron microscopy and/or mercury porosimetry, of 20 μm to 1000 μm, in particular 50 μm to 500 μm, preferably 100 μm to 300 μm. The pore diameters disclosed in this paragraph are particularly advantageous with respect to osteointegration of the encasing body.

Furthermore, pores, in particular surface pores, of the encasing body can be filled with an in vivo degradable/in vivo resorbable material. The pores can, in particular, be only partially or completely filled with the in vivo degradable/in vivo resorbable material. Preferably, the in vivo degradable/in vivo resorbable material which fills the pores, in particular surface pores, of the encasing body and the in vivo degradable/in vivo resorbable material of the enclosing body differ from one another. As a result, multistage, for example two-stage, degradation or resorption kinetics of the bone implant can be formed. This is advantageously a further way of balancing the mechanical stability of the bone implant, bone stimulation and bone growth in relation to one another. With regard to suitable in vivo degradable/in vivo resorbable materials, reference is made to the in vivo degradable/in vivo resorbable materials disclosed in the description that follows in connection with the encasing body.

Furthermore, the microstructured surface of the encasing body can have depressions with a depth of 20 μm to 1000 μm, in particular 100 μm to 600 μm, preferably 300 μm to 400 μm, and/or elevations with a height of 20 μm to 1000 μm, in particular 100 μm to 600 μm, preferably 300 μm to 400 μm.

Furthermore, the surface of the encasing body can be formed by a coating of the encasing body. In other words, the surface of the encasing body can be a surface of a coating which coats or covers the encasing body. The coating can at least partially, in particular only partially or completely, coat or cover the encasing body. Furthermore, the coating can have a thickness of 0.01 μm to 1000 μm, in particular 1 μm to 100 μm, preferably 10 μm to 50 μm. Furthermore, the coating can have a porosity of 20% to 90%, in particular 50% to 85%, preferably 60% to 80%. With regard to suitable materials for the coating, reference is made to the materials described in what is still to follow in connection with the encasing body.

In a further embodiment of the invention, the shaped bodies or only some of the shaped bodies have a porous, in particular open-pored, and/or microstructured surface.

In particular, the porous, in particular open-pored, surface of the shaped bodies can have pores having a diameter, in particular average diameter, determined by means of scanning electron microscopy and/or mercury porosimetry, of 50 μm to 900 μm, in particular 150 μm to 700 μm, particularly preferably 300 μm to 400 μm. The microstructured surface of the shaped bodies can have depressions with a depth of 50 μm to 900 μm, in particular 150 μm to 700 μm, preferably 300 μm to 400 μm, and/or elevations with a height of 50 μm to 900 μm, in particular 150 μm to 700 μm, preferably 300 μm to 400 μm. As a result, osteointegration and also, depending on the material of the shaped bodies, biological reconstruction and thus size reduction of a bone defect to be treated or managed is (likewise) achievable with particular advantage.

Furthermore, pores, in particular surface pores, of the shaped bodies can be filled with an in vivo degradable/in vivo resorbable material. The pores can, in particular, be only partially or completely filled with the in vivo degradable/in vivo resorbable material. Preferably, the in vivo degradable/in vivo resorbable material which fills the pores, in particular surface pores, of the shaped bodies and the in vivo degradable/in vivo resorbable material of the shaped bodies differ from one another. As a result, multistage, for example two-stage, degradation or resorption kinetics of the bone implant can be generated. This is advantageously a further way of balancing the mechanical stability of the bone implant, bone stimulation and bone growth in relation to one another. With regard to suitable in vivo degradable/in vivo resorbable materials, reference is made to the in vivo degradable/in vivo resorbable materials disclosed in the description that follows in connection with the shaped bodies.

Furthermore, the porous, in particular open-pored, surface of the shaped bodies or some of the shaped bodies can be formed by a coating of the shaped bodies. The coating can at least partially, in particular only partially or completely, coat or cover the shaped bodies. Furthermore, the coating can have a thickness of 0.01 μm to 1000 μm, in particular 1 μm to 100 μm, preferably 10 μm to 50 μm. Furthermore, the coating can have a porosity of 20% to 90%, in particular 50% to 85%, preferably 50% to 70%. With regard to further suitable materials for the coating, reference is made to the materials described in what is still to follow in connection with the shaped bodies.

Furthermore, the microstructured surface of the shaped bodies or some of the shaped bodies can have depressions with a depth of 50 μm to 900 μm, in particular 150 μm to 700 μm, preferably 300 μm to 400 μm, and/or elevations with a height of 50 μm to 900 μm, in particular 150 μm to 700 μm, preferably 300 μm to 400 μm.

Furthermore, the shaped bodies can have a thickness, i.e., wall thickness or height, in particular a constant, i.e., uniform, or varying, i.e., nonuniform, thickness, i.e., wall thickness or height, of 1 mm to 30 mm, in particular 5 mm to 20 mm, preferably 8 mm to 15 mm.

Advantageously, the total wall thickness of the bone implant can be controlled in a specific and thus application-oriented manner both via the thickness of the shaped bodies or the thickness of the encasing body and via the wall thickness of the main body or hollow body.

Furthermore, the shaped bodies are preferably in the form of sheets, i.e., the shaped bodies preferably have a length and a width that are greater than a thickness (height) of the shaped bodies. Preferably, the shaped bodies are in plate form and/or disk form. In particular, the shaped bodies can each have two main faces arranged opposite one another, each of which is delimited by two transverse side faces arranged opposite one another and by two longitudinal side faces arranged opposite one another. In principle, the transverse side faces can be planar, i.e. flat or not curved, and/or curved, in particular outwardly curved. Furthermore, in principle, the longitudinal side faces can be planar, i.e. flat or not curved, and/or curved, in particular outwardly curved. For example, the shaped bodies can each have one curved, in particular outwardly curved, longitudinal side face and, apart from that, planar side faces, i.e., one planar longitudinal side face and two planar transverse side faces.

Furthermore, the shaped bodies or some of the shaped bodies can each have at least one predetermined breaking line, in particular only one predetermined breaking line or a multiplicity of predetermined breaking lines. The predetermined breaking line(s) preferably extend(s) in the longitudinal direction of the shaped bodies. The predetermined breaking line(s) can, for example, be in the form of perforations, notches or scratches. As a result, additional adaptation of the shaped bodies and thus the bone implant to patient-specific bone defects is achievable. In particular, this can (additionally) reduce or completely avoid bone loss due to adaptation of a bone defect to the bone implant.

In a further embodiment of the invention, the encasing body comprises at least two different in vivo degradable/in vivo resorbable materials or consists of at least two different in vivo degradable/in vivo resorbable materials, which materials differ from one another with regard to their in vivo degradation rate/in vivo resorption rate. As a result, additional temporal control of the mechanical stability of the bone implant and of biological reconstruction of a bone defect is achievable. In particular, this can balance the mechanical stability of the bone implant, bone stimulation and bone growth in relation to one another even better.

In a further embodiment of the invention, the shaped bodies or some of the shaped bodies each comprise at least two different in vivo degradable/in vivo resorbable materials or the shaped bodies or some of the shaped bodies each consist of at least two different in vivo degradable/in vivo resorbable materials, which materials differ from one another with regard to their in vivo degradation rate/in vivo resorption rate. As a result, additional temporal control of the mechanical stability of the bone implant and of biological reconstruction of a bone defect is (likewise) achievable. In particular, this can balance the mechanical stability of the bone implant, bone stimulation and bone growth in relation to one another even better.

In a further embodiment of the invention, the in vivo degradable/in vivo resorbable material, in particular the at least two different in vivo degradable/in vivo resorbable materials, is/are selected from the group consisting of metals, polyhydroxyalkanoates, polycaprolactone, poly-p-dioxanone (poly-1,4-dioxan-2-one), polymethylene terephthalate, calcium phosphates, calcium sulfates, calcium oxide (CaO), calcium hydroxide (Ca(OH)₂), calcium carbonate (CaCO₃), calcium citrate, calcium lactate, calcium acetate, calcium tartrate, calcium chloride (CaCl₂), magnesium phosphates, magnesium sulfates, magnesium hydroxide (Mg(OH)₂), magnesium hydroxide carbonate (e.g., as 4 MgCO₃×Mg(OH)₂×5 H₂O), magnesium oxide (MgO), magnesium citrates (Mg₃(C₆H₅O₇)₂) or Mg(C₆H₆O₇)), calcium magnesium carbonate (CaMg(CO₃)₂ and combinations, in particular mixtures, thereof.

The metals are preferably magnesium.

The polyhydroxyalkanoates can, in particular, be selected from the group consisting of polylactide, polyglycolide, poly-3-hydroxybutyrate, poly-4-hydroxybutyrate, copolymers thereof and combinations, in particular mixtures (blends), thereof.

The calcium phosphates can, in particular, be selected from the group consisting of tricalcium phosphate (TCP) such as α-tricalcium phosphate (α-TCP) and/or β-tricalcium phosphate (β-TCP), apatites such as hydroxyapatite (HA), calcium-deficient hydroxyapatite (CdHA), substituted hydroxyapatite, nonstoichiometric hydroxyapatite, nanoscale hydroxyapatite, tetracalcium phosphate (TTCP), monocalcium phosphate monohydrate (MCPM), monocalcium phosphate anhydride (MCPA), dicalcium phosphate anhydride (DCPA), dicalcium phosphate dihydrate (DCPD), octacalcium phosphate (OCP), amorphous calcium phosphate (ACP), calcium glycerophosphate and combinations, in particular mixtures, thereof.

The calcium sulfates can, in particular, be selected from the group consisting of calcium sulfate (CaSO₄), calcium sulfate hemihydrate (CaSO₄×0.5H₂O), calcium sulfate dihydrate (CaSO₄×2 H₂O) and combinations, in particular mixtures, thereof.

The magnesium phosphates can, in particular, be selected from the group consisting of magnesium hydrogenphosphate (MgHPO₄) in the form of hydrates or as an anhydrous substance, trimagnesium phosphate (Mg₃(PO₄)₂), magnesium dihydrogenphosphate (Mg(H₂PO₄)₂) in the form of hydrates or as an anhydrous substance, magnesium glycerophosphate and combinations, in particular mixtures, thereof.

Particularly preferably, the in vivo degradable/in vivo resorbable material is tricalcium phosphate. The tricalcium phosphate can be in crystalline form. In particular, the tricalcium phosphate can have a crystallinity of 50% to 99%, in particular 75% to 95%. Furthermore, the tricalcium phosphate can be a microcrystalline tricalcium phosphate, i.e., tricalcium phosphate with crystallites having at least one dimension in the micrometer range, in particular within a range of >0.5 μm, in particular 0.6 μm to 500 μm, preferably 0.6 μm to 100 μm. The at least one dimension can be the length and/or the width and/or the thickness or height and/or the diameter, in particular average diameter, of the crystallites. Alternatively, the tricalcium phosphate can be a nanocrystalline tricalcium phosphate, i.e., tricalcium phosphate with crystallites having at least one dimension in the nanometer range, in particular within a range of 0.1 nm to 500 nm, preferably 0.1 nm to 100 nm. The at least one dimension can be the length and/or the width and/or the thickness or height and/or the diameter, in particular average diameter, of the crystallites. Alternatively, the tricalcium phosphate can be a macrocrystalline tricalcium phosphate. Alternatively, the tricalcium phosphate can be in amorphous form. Furthermore, the tricalcium phosphate can be a phase-pure tricalcium phosphate. Here, the expression “phase-pure” is to be understood in particular to mean phase-pure in the context of a relevant specification, preferably according to ASTM F1088. Furthermore, the tricalcium phosphate can have a porosity of <50%, in particular <20%, preferably <15%. Alternatively, the tricalcium phosphate can be nonporous. Preferably, the tricalcium phosphate is selected from the group consisting of α-tricalcium phosphate (α-TCP), β-tricalcium phosphate (β-TCP), and a mixture thereof. Especially preferably, the tricalcium phosphate is a sintered tricalcium phosphate, preferably selected from the group consisting of sintered α-tricalcium phosphate (sintered α-TCP), sintered β-tricalcium phosphate (sintered β-TCP) and a mixture thereof.

Alternatively, the in vivo degradable/in vivo resorbable material can, in particular, be hydroxyapatite. The hydroxyapatite can be in crystalline form. In particular, the hydroxyapatite can have a crystallinity of 50% to 99%, in particular 75% to 95%. Furthermore, the hydroxyapatite can be a microcrystalline hydroxyapatite, i.e., a hydroxyapatite with crystallites having at least one dimension in the micrometer range, in particular in a range of >0.5 μm, in particular 0.6 μm to 500 μm, preferably 0.6 μm to 100 μm. The at least one dimension can be the length and/or the width and/or the thickness or height and/or the diameter, in particular average diameter, of the crystallites. Alternatively, the hydroxyapatite can be a nanocrystalline hydroxyapatite, i.e., a hydroxyapatite with crystallites having at least one dimension in the nanometer range, in particular in a range of 0.1 nm to 500 nm, preferably 0.1 nm to 100 nm. The at least one dimension can be the length and/or the width and/or the thickness or height and/or the diameter, in particular average diameter, of the crystallites. Alternatively, the hydroxyapatite can be a macrocrystalline hydroxyapatite. Furthermore, the hydroxyapatite can be a phase-pure hydroxyapatite. In this connection, the expression “phase-pure” is to be understood in particular to mean phase-pure in the context of a relevant specification, preferably according to ASTM F1185. Furthermore, the hydroxyapatite can have a porosity of <50%, in particular <20%, preferably <15%. A low porosity means that a high mechanical stability can be achieved. Alternatively, the hydroxyapatite can be nonporous. Furthermore, the hydroxyapatite can, in particular, be a sintered hydroxyapatite.

Furthermore, the encasing body and/or the shaped bodies or some of the shaped bodies can comprise an in vivo nondegradable/in vivo nonresorbable material or consist of such a material. With regard to suitable in vivo nondegradable/in vivo nonresorbable materials, reference is made to the above materials mentioned in connection with the main body or hollow body.

Furthermore, the encasing body and/or the shaped bodies or some of the shaped bodies can comprise both an in vivo degradable/in vivo resorbable material and an in vivo nondegradable/in vivo nonresorbable material or consists of both an in vivo degradable/in vivo material resorbable material and an in vivo nondegradable/in vivo nonresorbable material. With regard to suitable in vivo degradable/in vivo resorbable materials and vivo nondegradable/in vivo nonresorbable materials, full reference is made to the disclosed materials that have been described above.

Furthermore, the bone implant can be produced by means of an additive manufacturing process. In particular, the encasing body and/or the shaped bodies can be shaped or joined on the main body or hollow body on the outside, i.e., on an outer face of the main body or hollow body, by means of an additive manufacturing process. With regard to further features and advantages of such a process, full reference is made to the following statements made in the context of the second aspect of the invention.

According to a second aspect, the invention provides a method for producing a bone implant, in particular a bone implant according to the first aspect of the invention.

The method involves producing the bone implant by means of an additive manufacturing process, i.e., by means of 3D printing.

As an alternative or in combination, a main body in the form of a hollow body open or opened on both sides in the axial direction, which main body comprises a load-bearing material or consists of a load-bearing material, is, by means of the/an additive manufacturing process, i.e., by means of 3D printing, at least partially, in particular only partially or completely, encased, in particular integrally encased, by an encasing body comprising or consisting of an in vivo degradable/in vivo resorbable material or connected, in particular integrally connected, to a multiplicity of shaped bodies comprising an in vivo degradable/in vivo resorbable material or consisting of an in vivo degradable/in vivo resorbable material in such a way that the shaped bodies protrude or project from the main body in the radial direction. The main body or hollow body is preferably conical.

The expression “additive manufacturing process” or “3D printing” is to be understood in the context of the present invention to mean processes for rapid and cost-effective manufacture of models, specimens, prototypes, tools, semifinished products and end products such as, in particular, bone implants. These processes are often also referred to as “rapid prototyping”. Manufacturing is directly carried out on the basis of internal computer data models from, in particular, shapeless material, for example from liquids, melts, powders or the like, or shape-neutral material, for example from material in tape form or wire form, by means of chemical and/or physical processes.

An additive manufacturing process offers, in particular, the advantage of specifically influencing the bone implant with regard to materials and/or shape and/or geometry and/or surface structure.

The additive manufacturing process can, in particular, be selected from the group consisting of powder bed process, free space process and liquid material process.

The powder bed process can be selected from the group consisting of selective laser melting, selective laser sintering, selective heat sintering, binder jetting and electron beam melting.

The free space process can be selected from the group consisting of fused deposition modeling, LOM process (laminated object modeling process), cladding, wax deposition modeling, contour grafting, cold spraying and electron beam melting.

The liquid material process can be selected from the group consisting of stereolithography, DLP process (digital light processing process) and LCM process. The LCM process can be a liquid composite molding process or a lithography-based ceramic manufacturing process.

With regard to further features and advantages of the method, in particular the bone implant, the main body, the encasing body and the shaped body, full reference is made to the statements made in the context of the first aspect of the invention. The statements made there in this respect also apply mutatis mutandis to the method according to the second aspect of the invention.

### BRIEF DESCRIPTION OF THE DRAWINGS

Further features and advantages of the invention will become apparent from the claims and from the following description of preferred exemplary embodiments of the invention, which are explained below with reference to the figures, where:

FIG. 1 schematically shows one embodiment of a bone implant according to the invention,

FIG. 2 schematically shows a further embodiment of a bone implant according to the invention and

FIG. 3 schematically shows a further embodiment of a bone implant according to the invention.

### DETAILED DESCRIPTION

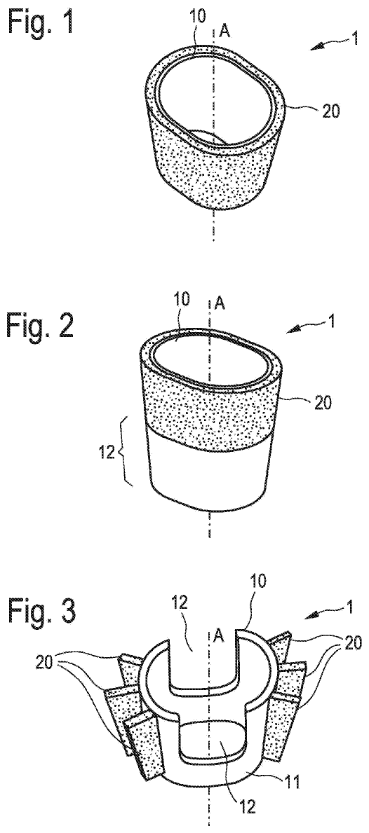

The bone implant 1 depicted schematically in FIG. 1 comprises a main body 10 and an encasing body 20.

The main body 10 is in the form of a hollow body which is open or opened on both sides in the axial direction A and which is preferably conical.

The main body 10 comprises a load-bearing material or consists of a load-bearing material. The load-bearing material is preferably an in vivo nondegradable/in vivo nonresorbable material, such as, for example, a titanium alloy or a plastic, such as, for example, polyetheretherketone. As a result, sufficient primary and secondary stability of the bone implant 1, after it has been placed on/in a bone defect, is achievable with particular advantage. With regard to further suitable materials for the main body 10, reference is made to the general description.

Preferably, as depicted in FIG. 1, the main body 10 is completely surrounded or encased by the encasing body 20. The encasing body 20 comprises an in vivo degradable/in vivo resorbable material or consists of such a material. The in vivo degradable/in vivo resorbable material can, for example, be a calcium phosphate, in particular β-tricalcium phosphate and/or hydroxyapatite. Alternatively, the in vivo degradable/in vivo resorbable material can be a polymer, such as, for example, polycaprolactone, or a metal, such as, for example, magnesium. With regard to further suitable materials for the encasing body 20, reference is made to the general description.

Owing to the (at least partially) in vivo degradable/in vivo resorbable encasing body 20, biological reconstruction of a bone defect to be treated or managed, such as, for example, a tibial or femoral bone defect, is achievable with particular advantage. It is especially advantageous that the size of the bone defect can be minimized by the biological reconstruction. This reduces the risk of any revision surgery. Owing to the reduction in the size of the bone defect, more favorable starting conditions may be created for successful performance of revision surgery.

The main body 10 can, for example, have a wall thickness of 3 mm to 5 mm, whereas the encasing body 20 can, for example, have a casing thickness of 2 mm to 10 mm.

As depicted in FIG. 1, the encasing body 20 can have a uniform thickness. Alternatively, the encasing body can have a nonuniform thickness (not depicted), in particular in the axial direction or longitudinal direction. As a result, asymmetrical bone defect structures can, in particular, be taken into account.

Furthermore, the encasing body 20 can have an open-pored surface. Alternatively, the encasing body 20 can be completely open-pored or be open-pored throughout. Owing to the two aforementioned embodiments for the encasing body 20, osteointegration, i.e., take and/or ingrowth of bone tissue on and/or into the encasing body 20, is achievable with particular advantage. This in turn contributes to an additional improvement in the primary and secondary stability of the bone implant 1.

Furthermore, the encasing body, in particular regions or layers thereof, can comprise different in vivo degradable/in vivo resorbable materials which differ from one another with regard to their in vivo degradation rate/in vivo resorption rate. As a result, different degradation/resorption kinetics of the encasing body 20 and, in particular, control of the mechanical stability of the bone implant 1, of bone stimulation and of bone growth are possible with particular advantage.

FIG. 2 shows a further embodiment of a bone implant 1 according to the invention.

The bone implant 1 comprises a main body 10 and an encasing body 20 which only partially surrounds the main body 10.

The main body 10 is in the form of a hollow body which is open or opened on both sides in the axial direction A and which is preferably conical. The main body 10 comprises a load-bearing material or consists of a load-bearing material. The load-bearing material is preferably an in vivo nondegradable/in vivo nonresorbable material, such as, for example, a titanium alloy, in particular the titanium alloy Ti-6Al-4V. This is a titanium alloy which comprises titanium, aluminum and vanadium and optionally impurities, in particular in the form of iron and/or oxygen and/or carbon and/or nitrogen and/or hydrogen, or consists of titanium, aluminum and vanadium and optionally impurities, in particular in the form of iron and/or oxygen and/or carbon and/or nitrogen and/or hydrogen. Preferably, the titanium alloy comprises, besides titanium, a proportion of aluminum of 5.5 percent by mass to 6.75 percent by mass, in particular 6 percent by mass, a proportion of vanadium of 3.5 percent by mass to 4.5 percent by mass, in particular 4 percent by mass, and optionally a proportion of iron of 0.4 percent by mass and/or a proportion of oxygen of 0.2 percent by mass and/or a proportion of carbon of 0.08 percent by mass and/or a proportion of nitrogen of 0.05 percent by mass and/or a proportion of hydrogen of 0.015 percent by mass or consists of the aforementioned elements/constituents. In particular, the main body 10 can have a lattice structure, preferably composed of Ti-6Al-4V. With regard to further suitable materials for the main body 10, reference is made to the general description and to the description of FIG. 1.

Owing to only partial encasing of the main body 10 by the encasing body 20, it is advantageously possible for the thickness of the bone implant 1 to be controlled in an application-oriented manner. Preferably, the nonencased region 12 of the bone implant 1 is intended to receive an extension shaft (not shown) of a joint implant. If the bone implant 1 is a tibial bone implant, the nonencased section 12 is a proximal section of the bone implant. By contrast, if the bone implant 1 is a femoral bone implant, the nonencased section 12 is a distal section of the bone implant.

With regard to further features and advantages of the bone implant 1, in particular the main body 10 and the encasing body 20, full reference is made to the description of FIG. 1 so as to avoid repetition. The features and advantages described there with regard to the bone implant 1, in particular the main body 10 and the encasing body 20, also apply mutatis mutandis to the bone implant 1 depicted in FIG. 2.

FIG. 3 shows a further embodiment of a bone implant 1 according to the invention.

The bone implant system 1 schematically depicted in FIG. 3 comprises a main body 10 and a multiplicity of shaped bodies 20. Six shaped bodies 20 are depicted by way of example. It will be appreciated that the bone implant 1 can, however, also comprise fewer shaped bodies or more shaped bodies.

The main body 10 is in the form of a conical hollow body open or opened on both sides in the axial direction A.

The main body 10 comprises a load-bearing material, in particular a titanium alloy, such as, for example, Ti-6Al-4V, or a plastic, in particular polyetheretherketone, or consists of such a material. As a result, sufficient primary and secondary stability is achievable with particular advantage. With regard to further suitable materials for the main body 10, reference is made to the general description and to the description of FIGS. 1 and 2.

Preferably, the main body 10 comprises a wall 11 having two preferably opposing openings or cutouts 12. The openings or cutouts 12 are preferably U-shaped.

The shaped bodies 20 comprise an in vivo degradable/in vivo resorbable material or consist of such a material. As a result, biological reconstruction of a bone defect can be achieved with particular advantage. The biological reconstruction of the bone defect advantageously leads to minimization of the size of the bone defect. This in turn reduces the risk of revision surgery and/or creates favorable conditions for successful performance of revision surgery. The in vivo degradable/in vivo resorbable material can, for example, be a calcium phosphate, in particular β-tricalcium phosphate and/or hydroxyapatite. Alternatively, the in vivo degradable/in vivo resorbable material can be a polymer, such as, for example, polycaprolactone, or a metal, such as, for example, magnesium. With regard to further suitable materials for the encasing body 20, reference is made to the general description and to the description of FIGS. 1 and 2.

The shaped bodies 20 protrude from the main body 10 in the radial direction. The shaped bodies 20 are integrally connected to the main body 10 or integrally joined thereto. For example, the shaped bodies 20 can be adhesively bonded or welded to the main body 10. Alternatively, the shaped bodies 20 can be shaped on the outside, i.e., on an outer face 12 of the main body 10, by means of an additive manufacturing process.

The shaped bodies 20 are preferably in the form of sheets, in particular in the form of plates or disks. This means that the shaped bodies 20 have a length and a width that are smaller than a thickness (height) of the shaped bodies 20. Preferably, the shaped bodies 20 each have two main faces arranged opposite one another, each of which is delimited by two transverse side faces arranged opposite one another and by two longitudinal side faces arranged opposite one another.

To improve osteointegration of the bone implant 1, it may further be preferred if the shaped bodies 20 each have a porous, in particular open-pored, and/or microstructured surface or consist of a porous, in particular open-pored, material. With regard to a material that is suitable in this respect, reference is likewise made to the general description and to the description of FIGS. 1 and 2.

1. A bone implant comprising:
a main body comprising a hollow body open on both sides in an axial direction, the main body comprising a load-bearing material; and
an encasing body which at least partially encases an outside of the main body, the encasing body comprising at least one in vivo degradable/in vivo resorbable material, or a plurality of shaped bodies which protrude from the main body in a radial direction and comprise the at least one in vivo degradable/in vivo resorbable material.

2. The bone implant according to claim 1, wherein the main body has a corner-free cross section.

3. The bone implant according to claim 1, wherein the main body is conical and has an at least sectionally tapering inner diameter.

4. The bone implant according to claim 1, wherein the main body has a wall thickness of 1 mm to 30 mm.

5. The bone implant according to claim 1, wherein the load-bearing material is an in vivo nondegradable/in vivo nonresorbable material.

6. The bone implant according to claim 1, wherein the load-bearing material is selected from the group consisting of metals, alloys, ceramics, plastics, and combinations thereof.

7. The bone implant according to claim 1, wherein the encasing body comprises a layer or coating on the main body.

8. The bone implant according to claim 1, wherein the encasing body has a thickness of 0.1 mm to 30 mm.

9. The bone implant according to claim 1, wherein the encasing body has a nonuniform shape and/or a non-uniform thickness in the axial direction.

10. The bone implant according to claim 1,
wherein the encasing body has a thickness,
the thickness being a first thickness at a first end of the encasing body and a second thickness at a second end of the encasing body, and
wherein at least one of:
the first thickness is greater than the second thickness; and
the thickness of the encasing body increases toward the first end of the encasing body.

11. The bone implant according to claim 1, wherein the main body is partially exposed and not encased by the encasing body on the outside of the main body.

12. The bone implant according to claim 1, wherein at least one of the main body and the encasing body comprises a porous and/or microstructured surface.

13. The bone implant according to claim 1, wherein the at least one in vivo degradable/in vivo resorbable material comprises at least two in vivo degradable/in vivo resorbable materials which differ from one another with regard to their in vivo degradation rate/in vivo resorption rate, or the shaped bodies each comprise the at least two in vivo degradable/in vivo resorbable materials which differ from one another with regard to their in vivo degradation rate/in vivo resorption rate.

14. The bone implant according to claim 1, wherein the at least one in vivo degradable/in vivo resorbable material is selected from the group consisting of polyhydroxyalkanoates, calcium phosphates and combinations thereof.

15. A method for producing the bone implant according to claim 1, comprising the steps of:
producing the bone implant by an additive manufacturing process; and/or
producing the main body by the additive manufacturing process, and at least partially encasing the main body with the encasing body.

16. The bone implant according to claim 1, wherein the main body has a circular, oval or elliptical cross section.

17. The bone implant according to claim 1, wherein the load-bearing material is selected from the group consisting of titanium, tantalum, titanium alloys, polyetherketones, polyetheretherketone, polyetherketoneketone, polyetheretheretherketone, polyetheretherketoneketone, polyetherketoneetherketoneketone, polyolefins and combinations thereof.

18. The bone implant according to claim 1, wherein the main body and/or the encasing body and/or the shaped bodies have an open-pored surface.

19. The bone implant according to claim 1, wherein the in vivo degradable/in vivo resorbable material is selected from the group consisting of polylactide, polyglycolide, polycaprolactone, β-tricalcium phosphate, hydroxyapatite and combinations thereof.

20. The bone implant according to claim 13, wherein the at least two in vivo degradable/in vivo resorbable materials are selected from the group consisting of polyhydroxyalkanoates, calcium phosphates and combinations thereof.
